(19) United States
(12) Patent Application Publication
(10) Pub. No.: US 2018/0049878 A1
(43) Pub. Date: Feb. 22, 2018

(54) AUGMENTS FOR BONE DEFICIENCIES

(71) Applicant: EXACTECH, INC., Gainesville, FL (US)

(72) Inventors: Bernard STULBERG, Gainesville, FL (US); Laurent ANGLBAUD, Gainesville, FL (US); Luis ALVAREZ, Gainesville, FL (US); Jim DOYLE, Gainesville, FL (US)

(21) Appl. No.: 15/554,189

(22) PCT Filed: Jan. 7, 2016
(86) PCT No.: PCT/US16/12491

§ 371 Date: Aug. 28, 2017

Related U.S. Application Data
(60) Provisional application No. 62/100,626, filed on Jan. 7, 2015.

(51) Int. Cl. A61F 2/30; A61F 2/38

(52) U.S. Cl. CPC A61F 2/30734; A61F 2002/2835; A61F 2002/30736; A61F 2/389

(57) ABSTRACT

In one embodiment, the present invention provides an augment comprising: a. a side configured to contact and attach to a receiving bone; b. a side configured to contact an implant; and c. an exterior profile, wherein the side configured to contact a receiving bone and the side configured to contact an implant are separated by a thickness; and wherein the augment is configured to anchor to the receiving bone.

### CROSS REFERENCE TO RELATED APPLICATIONS

This application claims priority to U.S. Provisional Patent Application Ser. No. 62/100,626, filed on Jan. 7, 2015, the contents of which are incorporated by reference in its entirety.

### FIELD

The present invention relates to augments for bone deficiencies and a procedure for implanting these augments. The augments of the present invention, when implanted in the body, are sufficiently designed to replace joint stability and function to the extent possible, and to restore deficient bone to enhance that ability.

### BACKGROUND

Implantation of joint-replacement prostheses, for example in total knee or total hip replacement operations, sometimes requires compensation for bone loss. For example, peripheral defects in the proximal tibia can be difficult to treat during total knee arthroplasty. Conventionally, attempts can be made to solve the problem with cement, cement with screws, bone grafts, metal wedges, metal cones, metal sleeves and custom components. These “augments” provide support to the main implant, fashion a specific custom implant for a particular patient's deficiencies, or provide a technique of removing bone to a predictable shape, that allows some standardization of the augments and the technique for implanting them. Augments allow positioning of joint implants in a kinematically appropriate manner. Remaining deficiencies or gaps are filled either with bone graft, bone cement or both. The metal augments are initially attached directly to the implant, using a number of modes of attachment, including screws, snap-fit mating geometry and bone cement, and then the composite (augment/implant) is attached to bone either directly or with bone cement.

Current trends in joint replacement show mean patient age is decreasing as overall life expectancy increases, so it can be concluded that some patients will most likely require multiple surgeries throughout their lives. Therefore, maintaining as much patient bone stock as possible, minimizing any bone loss due to revision procedures, is likely beneficial. Recurring failure and the progressive loss of bone support for implant components is more easily understood in the scenario of the multiply failed total knee arthroplasty, but the concept of progressive failure of current approaches is equally prevalent in failed hip, shoulder, and even small joint arthroplasty. If bone loss continues, the solutions have been to add more metal; either as augments to present devices (larger and larger augments added to tibial and femoral components, the addition of cones and or sleeves, etc.), or as replacements for bone (distal femoral replacements).

### SUMMARY

The present invention relates to augments for bone deficiencies and a procedure for implanting these augments. The augments of the present invention, when implanted in the body, are sufficiently designed to replace joint stability and function to the extent possible, and to restore deficient bone to enhance that ability.

According to aspects illustrated herein, an augment includes a first side (sufficiently designed to contact a receiving bone), a second side (sufficiently designed to contact an implant), and an exterior profile, wherein the first side and the second side are separated by a thickness, wherein the augment is sufficiently designed for anchoring to the receiving bone at a surface of the bone associated with the void to repair the bone defect. In an embodiment, the exterior profile of the augment is defined by a posterior curvature, a lateral curvature, an anterior curvature, and a truncated aspect. In an embodiment, the augment is manufactured from a metal biomaterial. In an embodiment, the augment is manufactured from a ceramic biomaterial. In an embodiment, the augment is manufactured from a composite biomaterial. In an embodiment, the augment is manufactured from a polymer biomaterial. In an embodiment, the augment is manufactured from a natural polymer biomaterial. In an embodiment, the augment is manufactured from a synthetic polymer biomaterial. In an embodiment, the augment is manufactured from a combination of a natural and synthetic polymer biomaterial. In an embodiment, a portion of the augment is manufactured from a resorbable biomaterial. In an embodiment, the entire augment is manufactured from a resorbable biomaterial. In an embodiment, the at least one fixation means is selected from the group consisting of pegs, pins, posts, buttons, keels, spikes and hooks.

According to aspects illustrated herein, an augment includes a three dimensional endoskeleton support having interconnected macropores providing a framework for host bone to grow into the augment, wherein the support has a first side (sufficiently designed to contact a receiving bone), a second side (sufficiently designed to contact an implant), and an exterior profile, wherein the first side and the second side are separated by a thickness, wherein the support is sufficiently designed for anchoring to the receiving bone at a surface of the bone associated with the void to repair the bone defect. In an embodiment, the exterior profile is defined by a posterior curvature, a lateral curvature, an anterior curvature, and a truncated aspect. In an embodiment, the support is manufactured from a natural biomaterial. In an embodiment, the support is manufactured from a synthetic biomaterial. In an embodiment, the support is manufactured from a combination of a natural and synthetic biomaterial. In an embodiment, a portion of the support is manufactured from a resorbable biomaterial. In an embodiment, the entire support is manufactured from a resorbable biomaterial. In an embodiment, the at least one fixation means is selected from the group consisting of pegs, pins, posts, buttons, keels, spikes and hooks.

According to aspects illustrated herein, an augment includes a three dimensional endoskeleton support having interconnected macropores providing a framework for host bone to grow into the augment, wherein the support is manufactured from a microporous biomaterial, wherein the support has a first side, a second side and an exterior profile defined by a posterior curvature, a lateral curvature, an anterior curvature, and a truncated aspect, wherein the first side and the second side are separated by a thickness, wherein the augment is sufficiently designed for anchoring to the receiving bone at a surface of the bone associated with the void to repair the bone defect, and wherein the second side is sufficiently designed to engage a component of an orthopaedic implant. In an embodiment, the support is manufactured from a natural biomaterial. In an embodiment, the support is manufactured from a synthetic biomaterial. In an embodiment, the support is manufactured from a combination of a natural and synthetic biomaterial. In an embodiment, a portion of the support is manufactured from a resorbable biomaterial. In an embodiment, the entire support is manufactured from a resorbable biomaterial. In an embodiment, the at least one fixation means is selected from the group consisting of pegs, pins, posts, buttons, keels, spikes and hooks.

According to aspects illustrated herein, a prosthesis system includes a joint prosthesis component; and an augment comprising a first side (sufficiently designed to contact a receiving bone), a second side (sufficiently designed to contact an implant), and thickness therebetween, wherein the augment is sufficiently designed for anchoring to the receiving bone at a surface of the bone associated with the void to repair the bone defect. In an embodiment, the system further includes bone graft material. In an embodiment, the augment is manufactured from a metal biomaterial. In an embodiment, the augment is manufactured from a ceramic biomaterial. In an embodiment, the augment is manufactured from a composite biomaterial. In an embodiment, the augment is manufactured from a polymer biomaterial. In an embodiment, the augment is manufactured from a natural polymer biomaterial. In an embodiment, the augment is manufactured from a synthetic polymer biomaterial. In an embodiment, the augment is manufactured from a combination of a natural and synthetic polymer biomaterial. In an embodiment, a portion of the augment is manufactured from a resorbable biomaterial. In an embodiment, the entire augment is manufactured from a resorbable biomaterial. In an embodiment, the at least one fixation means is selected from the group consisting of pegs, pins, posts, buttons, keels, spikes and hooks.

According to aspects illustrated herein, a knee prosthesis system includes a tibial tray component having an inferior surface, a superior surface, and an exterior profile; and an augment comprising a distal side (sufficiently designed to contact a receiving bone), a proximal side (sufficiently designed to contact an implant and an exterior profile, wherein the distal side and the proximal side are separated by a thickness, wherein the augment is sufficiently designed for anchoring to the receiving bone at a surface of the bone associated with the void to repair the bone defect. In an embodiment, the exterior profile is defined by a posterior curvature, a lateral curvature, an anterior curvature, and a truncated aspect, at least one of which mimics the exterior profile of the tibial tray component. In an embodiment, the system further includes bone graft material. In an embodiment, the augment is manufactured from a metal biomaterial. In an embodiment, the augment is manufactured from a ceramic biomaterial. In an embodiment, the augment is manufactured from a composite biomaterial. In an embodiment, the augment is manufactured from a polymer biomaterial. In an embodiment, the augment is manufactured from a natural polymer biomaterial. In an embodiment, the augment is manufactured from a synthetic polymer biomaterial. In an embodiment, the augment is manufactured from a combination of a natural and synthetic polymer biomaterial. In an embodiment, a portion of the augment is manufactured from a resorbable biomaterial. In an embodiment, the entire augment is manufactured from a resorbable biomaterial. In an embodiment, the at least one fixation means is selected from the group consisting of pegs, pins, posts, buttons, keels, spikes and hooks.

In one embodiment of the present invention, the prosthesis system is a kit.

According to aspects illustrated herein, a method includes assessing a bone defect of a tibial bone; implanting an augment within the bone defect so as to create a substantially flush proximal surface, wherein the augment comprises a distal side that engages a surface of the bone defect, a proximal side, and an exterior profile defined by a posterior curvature, a lateral curvature, an anterior curvature, and a truncated aspect, wherein the distal side and the proximal side are separated by a thickness, wherein the distal side includes at least one fixation means sufficiently designed to facilitate implanting the augment to a surface of the bone associated with the void to repair the bone defect; adding bone graft material to the augment; positioning a tibial tray component on top of the proximal side of the augment; and fixing the tibial tray component to the augment.

In one embodiment, the present invention provides a method, wherein the method restores bone support for an implant for joint arthroplasty, wherein the method comprises: a. assessing a defect of a bone, wherein the bone defect has a first void; b. preparing a first surface of the bone not associated with the first void; c. selecting a first individual augment from a kit, wherein the thickness of the first individual augment is sufficient to fill the first void; d. preparing a second surface of the bone associated with the first void and offset from the first surface by the thickness of the first individual augment; e. implanting the first individual augment within the second surface of the bone so the side configured to contact an implant of the first individual augment is substantially co-planar with the first surface of the bone; f positioning an implant on top of the reconstructed flat surface created by the first surface of the bone and the side configured to contact an implant of the first individual augment; and g. fixing the implant to the bone and the side configured to contact an implant of the first individual augment.

In one embodiment, bioactive bone graft material is added to the first individual augment.

In one embodiment, the first individual augment is used in a joint arthroplasty selected from the group consisting of: total knee arthroplasty, total hip arthroplasty, and total shoulder arthroplasty; or wherein the first individual augment is used to restore bone stock in a bone defect selected from the group consisting of: a femoral bone defect, an acetabular bone defect, a humeral bone defect, a glenoid bone defect, and a periprosthetic bone defect.

In one embodiment, the present invention provides a method, wherein the method restores bone support for an implant for joint arthroplasty, wherein the method comprises: a. assessing a defect of a bone, wherein the bone defect has a first and second void; b. selecting a first individual augment from a kit, wherein the thickness of the first individual augment is sufficient to fill the first void; c. preparing a first surface of the bone associated with the first void; d. selecting a second individual augment from the kit, wherein the thickness of the second individual augment is sufficient to fill the second void; e. preparing a second surface of the bone associated with the second void and offset from the first surface by the difference between the thickness of the first individual augment and the thickness of the second individual augment, f implanting the first individual augment within the first surface of the bone, and implanting the second individual augment within the second surface of the bone so the sides configured to contact an implant of the first and second individual augments are substantially co-planar; g. positioning an implant on top of the sides configured to contact an implant of the first and second individual augments; and h. fixing the implant to the sides configured to contact an implant of the first and second individual augments, wherein the exterior profiles of the first and second individual augments together substantially match an exterior profile of the implant.

In one embodiment, bioactive bone graft material is added to the first individual augment, or the second individual augment, or the first and second individual augments.

In one embodiment, the first and second individual augments are used in a joint arthroplasty selected from the group consisting of: total knee arthroplasty, total hip arthroplasty, and total shoulder arthroplasty; or wherein the first and second individual augments are used to restore bone stock in a bone defect selected from the group consisting of: a femoral bone defect, an acetabular bone defect, a humeral bone defect, a glenoid bone defect, and a periprosthetic bone defect.

In one embodiment, the present invention provides a method, wherein the method restores bone support for an implant for joint arthroplasty, wherein the method comprises: a. assessing a defect of a bone, wherein the bone defect has a first void; b. selecting at least one first individual augment from a kit, wherein the thickness of the at least one first individual augment is sufficient to fill the first void and provide a surface for an implant; c. preparing a first surface of the bone associated with the first void; d. implanting the at least one first individual augment within the first surface of the bone; e. positioning the implant on top of the side configured to contact an implant of the at least one first individual augment; and f. fixing the implant to the side configured to contact an implant of the at least one first individual augment, wherein the exterior profile of the at least one first individual augment substantially match an exterior profile of the implant.

In one embodiment, bioactive bone graft material is added to the at least one first individual augment.

In one embodiment, the at least one first individual augment is used in a joint arthroplasty selected from the group consisting of: total knee arthroplasty, total hip arthroplasty, and total shoulder arthroplasty; or wherein the at least one first individual augment is used to restore bone stock in a bone defect selected from the group consisting of: a femoral bone defect, an acetabular bone defect, a humeral bone defect, a glenoid bone defect, and a periprosthetic bone defect.

In an embodiment, the first individual augment is fixed to the bone by one of pegs, pins, posts, buttons, keels, spikes or hooks.

In an embodiment, the at least one first individual augment is fixed to the bone by one of pegs, pins, posts, buttons, keels, spikes or hooks.

In an embodiment, the second individual augment is fixed to the bone by one of pegs, pins, posts, buttons, keels, spikes or hooks.

In an embodiment, the augment is manufactured from a metal biomaterial. In an embodiment, the augment is manufactured from a ceramic biomaterial. In an embodiment, the augment is manufactured from a composite biomaterial. In an embodiment, the augment is manufactured from a polymer biomaterial. In an embodiment, the augment is manufactured from a natural polymer biomaterial. In an embodiment, the augment is manufactured from a synthetic polymer biomaterial. In an embodiment, the augment is manufactured from a combination of a natural and synthetic polymer biomaterial. In an embodiment, a portion of the augment is manufactured from a resorbable biomaterial. In an embodiment, the entire augment is manufactured from a resorbable biomaterial. In an embodiment, the at least one fixation means is selected from the group consisting of pegs, pins, posts, buttons, keels, spikes and hooks.

In an embodiment, the augments of the present invention combine the benefits of both shape matching technologies and new developments in materials and shape manufacturing to provide predictably resorbable biologically compatible materials for this intervention. The augments of the present disclosure may be manufactured from a biological material, a biologically resorbable material, or a combination of biological and biologically resorbable materials, and are porous structures. In an embodiment, the augment is manufactured from a metal biomaterial. In an embodiment, the augment is manufactured from a ceramic biomaterial. In an embodiment, the augment is manufactured from a composite biomaterial. In an embodiment, the augment is manufactured from a polymer biomaterial. In an embodiment, the augment is manufactured from a natural polymer biomaterial. In an embodiment, the augment is manufactured from a synthetic polymer biomaterial. In an embodiment, the augment is manufactured from a combination of a natural and synthetic polymer biomaterial. In an embodiment, a portion of the augment is manufactured from a resorbable biomaterial. In an embodiment, the entire augment is manufactured from a resorbable biomaterial.

In an embodiment, an augment of the present disclosure includes a three dimensional (3D) endoskeleton support for bioactive bone grafting material and is sufficiently designed to directly attach to host bone (not the implant) and enables permanent restoration of bone support for metallic implant devices that are used to restore stability and function to the damaged joint (e.g., a tibial tray). An augment of the present disclosure has a shape of which can be designed to fit a bone defect using anatomical information obtained from the patient. In an embodiment, an augment of the present invention has a wedge shape. In an embodiment, an augment of the present invention has a block shape.

An implanted augment of the present disclosure is sufficiently designed to restore and/or enhance local bone support for implants (“bone stock”) in situations of uncontained deficiencies (combined cortical and cancellous deficiencies that compromise implant support). An implanted augment of the present disclosure is sufficiently designed to restore bone while providing support to the mechanical implant (e.g., a tibial tray), and offers the potential to improve patient bone support for future implants, even if the device implanted at the time of this augmentation requires revision. This concept seeks to develop the independence of these two events, so that restoration of bone support becomes one of the main goals of the operative intervention, not simply maintenance.

### BRIEF DESCRIPTION OF THE DRAWINGS

The presently disclosed embodiments will be further explained with reference to the attached drawings.

FIG. 1 is a schematic illustration of a tibia with a bone defect on the lateral side.

FIG. 2 shows a black-and-white line drawing of an embodiment of an augment of the present invention.

FIG. 3 shows an embodiment of a tibial tray component of a knee prosthesis.

FIG. 4 shows a black-and-white line drawing of an embodiment of an augment with spikes of the present invention.

FIG. 5 shows a black-and-white line drawing of an embodiment of an augment with pegs of the present invention.

FIG. 6 shows a black-and-white line drawing of an embodiment of an augment with keel of the present invention.

FIG. 7 is a schematic illustration showing the implanting of the tibial tray component of FIG. 3 into a reconstructed proximal tibia having an augment of the present invention.

FIG. 8 shows an embodiment of an augment with spikes of the present invention for use in a distal femoral augmentation.

FIG. 9 shows an embodiment of an augment with spikes of the present invention for use in a distal femoral augmentation.

FIG. 10 shows an embodiment of an augment with spikes of the present invention for use in a distal femoral augmentation.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present invention relates to augments for bone deficiencies and a procedure for implanting these augments. In an embodiment, the augments of the present invention combine the benefits of both shape matching technologies and new developments in materials and shape manufacturing to provide predictably resorbable biologically compatible materials for this intervention. The augments of the present disclosure may be manufactured from a biomaterial, a resorbable biomaterial or a combination of biomaterial and resorbable biomaterial. In an embodiment, the augment is manufactured from a metal biomaterial. In an embodiment, the augment is manufactured from a ceramic biomaterial. In an embodiment, the augment is manufactured from a composite biomaterial. In an embodiment, the augment is manufactured from a polymer biomaterial. In an embodiment, the augment is manufactured from a natural polymer biomaterial. In an embodiment, the augment is manufactured from a synthetic polymer biomaterial. In an embodiment, the augment is manufactured from a combination of a natural and synthetic polymer biomaterial. In an embodiment, a portion of the augment is manufactured from a resorbable biomaterial. In an embodiment, the entire augment is manufactured from a resorbable biomaterial. In an embodiment, an augment of the present disclosure includes a three dimensional (3D) endoskeleton support for bioactive bone grafting material and is sufficiently designed to directly attach to host bone (not the implant) and enables permanent restoration of bone support for metallic implant devices that are used to restore stability and function to the damaged joint (e.g., a tibial tray). An augment of the present disclosure has a shape of which can be designed to fit a bone defect using anatomical information obtained from the patient.

In an embodiment, an augment of the present invention includes a three dimensional (3D) endoskeleton support for bioactive bone grafting and is manufactured from a biomaterial, a resorbable biomaterial or a combination of biomaterial and resorbable biomaterial. The biomaterial of the augment displays both mechanical resilience and provides an osteoconductive surface. Unlike conventional metal augments, the augments of the present invention are first attached to the host bone. The addition of the augment to the bone is intended to restore the native shape of the bone. Next, the surgeon will position the implant (e.g., a tibial tray) on top of the augment and contact the two. In the event of a future revision, it is expected the osteointegrated augment stays attached to the bone, resulting in no additional patient bone loss at the level of the augment. An augment of the present invention is available in a variety of sizes and shapes to accommodate defects of various sizes. Both the choice of the biomaterial and the design of the augment are defined in order to sustain the physiological loading pattern associated with the considered joint. An augment of the present invention is sufficiently designed to function as a load bearing implant. In an embodiment, an augment of the present invention has high tensile, high fatigue and high yield strengths. In an embodiment, an augment of the present invention has low reactivity.

As used herein, the term “biomaterial” refers to a synthetic or natural material appropriate for long term use in the body as a medical implant. Examples of such materials that are synthetic include, but are not limited to, polymers (e.g., PEEK or polyethylene), ceramics or bioglass materials (e.g., calcium phosphates or oxides, or carbon or graphite based materials such as pyrolitic carbon). Examples of such materials that are of natural origin include polymers such as polypeptides, including collagen materials derived from humans, animals, or recombinant organisms, which may be modified to reduce or eliminate degradation in the body, polysaccharides such as cellulosic materials, starches, or chitin, or materials consisting of or derived from the bone of human or animal origin or metals.

As used herein, the term “resorbable biomaterial” refers to the subset of “biomaterial” that can be a resorbable material, meaning a material that is designed to disintegrate in the body over time. Examples of such resorbable materials that are synthetic include polymers such as the polyesters PLGA and PLLA, polyanhydrides, polyorthoesters, or polyamides, or certain ceramic or oxide based materials such as calcium sulfates or bioglasses. Examples of such materials of natural origin include polymers such as polypeptides, including collagen materials derived from humans, animals, or recombinant organisms, polysaccharides, such as alginates, hyaluronan, or chitosan, including polysaccharides that are modified to enhance resorbability by processes such as oxidation.

As used herein, the term “bioactive bone graft material” refers to autologous material (e.g., bone harvested from the patient's own body, often from the iliac crest), allograft material (e.g., cadaveric bone usually obtained from a bone bank), or synthetic material (e.g., often made of hydroxyapatite or other naturally occurring and biocompatible substances) with similar mechanical properties to bone.

As used herein, the term “thickness” refers to the dimension between the distal surface and the proximal surface of an augment of the present invention. An augment can be of varying thickness in the range of approximately 1 mm to 30 mm, and preferably of 5 mm to about 11 mm. Desired thickness can be achieved by selecting or shaping a single augment to desired thickness, or by stacking multiple augments.

As used herein, the term “void” refers to a volume of bone loss or a bone defect in cancellous bone, cortical bone, or both cancellous and cortical bone.

In an embodiment, an augment of the present invention acts as a 3D endoskeleton support for bioactive bone graft material and includes interconnected “macropores”, typically of 100 μm to about 3 mm diameter. In an embodiment, the macropores range from about 1 mm-3 mm diameter. In an embodiment, the macropores range from about 100-1000 μm diameter. In an embodiment, the macropores range from about 150-900 μm diameter. In an embodiment, the macropores range from about 200-800 μm diameter. In an embodiment, the macropores range from about 300-700 μm diameter. These macropores provide a framework for the host bone to regenerate while reducing healing time. The macropores allow bone tissue to grow into the augment to yield cell/augment integration. The host's own bone tissue uses the macroporous structure to grow into the augment material, the material being slowly degraded and being replaced by new bone growth. Ideally, biomaterials used for the augments should be microporous with a pore diameter of 1-10 μm. In an embodiment, the micropores range from about 2-9 μm diameter. In an embodiment, the micropores range from about 3-8 μm diameter. In an embodiment, the micropores range from about 4-7 μm diameter. Such micropores have been found to improve the ability of osteoblasts and other cells from the host to bind to the biomaterial of the augment and to allow access of the cells to dissolve the sintered connections between the individual particles.

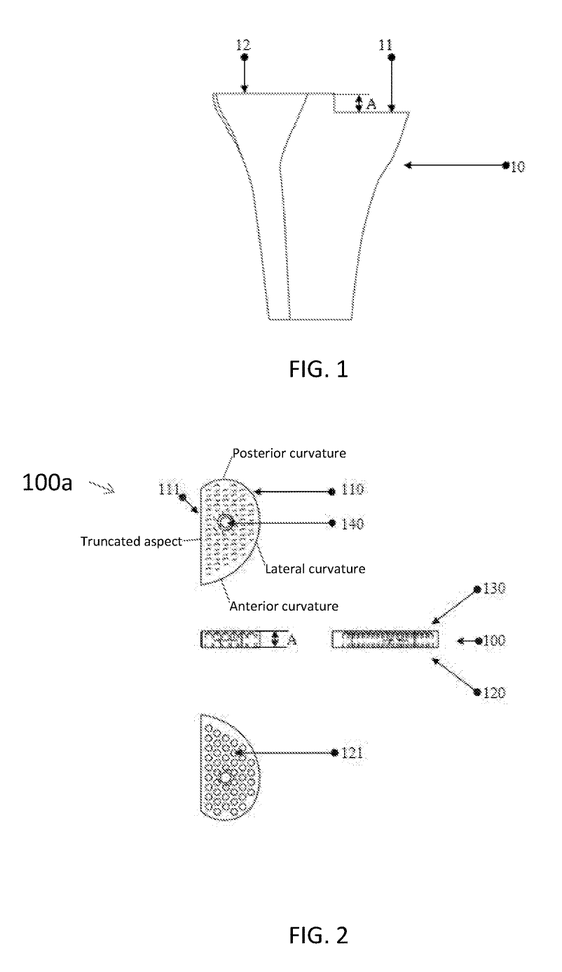

For illustration purposes only, assume a bone defect where the proximal tibia (10) has a defect on its lateral aspect (11) (see FIG. 1). The initial steps of the preparation are going to be as described above in steps 1 and 2, where the surgeon prepares the proximal tibia using a resection guide.

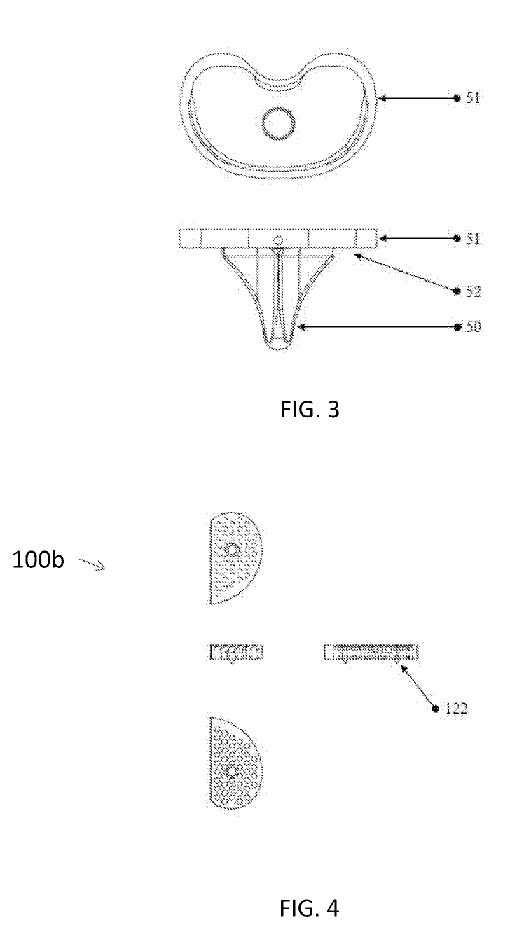

At this point, rather than attaching a metal augment to the prosthesis per the conventional technique (see left side of Table 1), the surgeon uses an augment of the present invention (100a, 100b, 100c or 100d) (see FIGS. 2, 4, 5 and 6) with overall dimensions more or less matching those of a standard conventional metal augment (see right side of Table 1). The exterior profile (110) of the augment (100a, 100b, 100c or 100d) is dimensionally equivalent to the outside profile (51) of a tibial tray component (50) (see FIG. 3) except for one aspect that is truncated (111). Usually the truncated aspect (111) is designed so the augment covers one half or one third of the tibial tray component (50) distal surface (52).

The augment (100a, 100b, 100c or 100d) features two opposite surfaces: a distal surface (120) intended to contact the proximal tibia at the surface of the defect (11) and a proximal surface (130) intended to contact the distal aspect (52) of the tibial tray component (50); where both surfaces are separated by a dimension (A; where usually A ranges from 5 mm to 20 mm) consistent with the thickness (A) of the void to be addressed in repairing the defect. The exterior profile (110) of the augment (100a, 100b, 100c or 100d) has a posterior curvature, a lateral curvature and an anterior curvature.

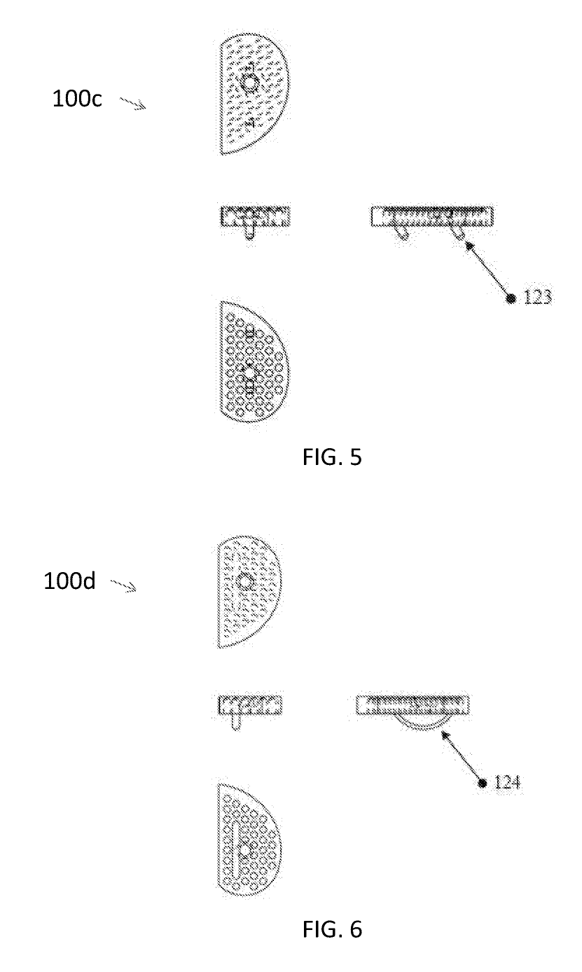

The distal surface (120) of the augment (100a, 100b, 100c or 100d) features a network of channels or openings (121) intended to receive bioactive bone graft material at the time of implantation. In addition, the distal surface may feature spikes (122) (see FIG. 4), pegs (123) (see FIG. 5), or a keel (124) (see FIG. 6) to facilitate anchoring with the receiving bone at the surface associated with the void (11) by improving the stability of the augment (100a, 100b, 100c or 100d) relative the bone (10) after implantation. The spikes, pegs and keel can be self-locking and provide resistance to anterior and posterior translation, liftoff, and subsidence. Other type of fasteners are acceptable, including, but not limited to, pins, posts, buttons, friction fasteners, sutures, and hooks.

The augment (100a, 100b, 100c or 100d) can include a countersink (140) intended to receive a fastener (e.g., locking screw or other type of device to allow proper anchorage) to lock the augment (100a, 100b, 100c or 100d) relative to the bone (10).

After tibial bone preparation is complete, the surgeon introduces the bioactive bone graft (not represented) into the network of channels or openings (121) of the augment (100a, 100b, 100c or 100d) up to saturation. Then the surgeon positions and impacts the augment (100a, 100b, 100c or 100d) into the bone defect (11). It is understood that depending on the size of the spikes (122) (see FIG. 4), pegs (123) (see FIG. 5), or keel (124) (see FIG. 6), pre-drilling or tamping may be needed to facilitate impaction. In an embodiment, the augment (100a, 100b, 100c or 100d) is seeded with living cells capable of regenerating bone. In an embodiment, the augment (100a, 100b, 100c or 100d) is seeded with living cells capable of regenerating bone in an amount sufficient to regenerate bone.

With the augment (100a, 100b, 100c or 100d) in place, its proximal surface (130) should be substantially flush with the healthier, more proximal surface of the proximal tibia (12). It is understood at this point the void associated with the bone defect (11) has been filled by the augment (100a, 100b, 100c or 100d).

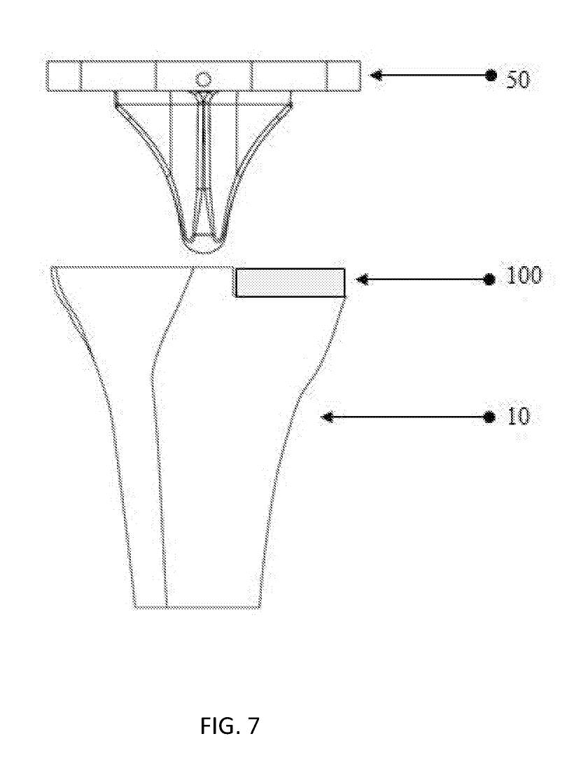

After this reconstruction is established, the surgeon can implant the tibial tray component (50) onto the reconstructed and now substantially co-planar surface (12 and 130) (see FIG. 7) using conventional PMMA bone cement or another type of material intended to fix two materials together (not represented). The other components of the knee prosthesis can then be implanted as known in the art.

The above illustrative example describes one embodiment wherein a single augment is used to restore bone support in a single void to repair a defect in a bone. In alternate embodiments of the present invention, at least one augment is used to restore bone support in a void to repair a single defect in a bone. In alternate embodiments, at least one augment is used to restore bone support in at least one void to repair a defect in a bone.

For example, for illustration purposes only, in some embodiments, the present invention provides a method, wherein the method restores bone support for an implant for joint arthroplasty, wherein the method comprises: a. assessing a defect of a bone, wherein the bone defect has a first void; b. preparing a first surface of the bone not associated with the first void; c. selecting a first individual augment from a kit, wherein the thickness of the first individual augment is sufficient to fill the first void; d. preparing a second surface of the bone associated with the first void and offset from the first surface by the thickness of the first individual augment; e. implanting the first individual augment within the second surface of the bone so the side configured to contact an implant of the first individual augment is substantially co-planar with the first surface of the bone; f. positioning an implant on top of the reconstructed flat surface created by the first surface of the bone and the side configured to contact an implant of the first individual augment; and g. fixing the implant to the bone and the side configured to contact an implant of the first individual augment.

In some embodiments, bioactive bone graft material is added to the first individual augment.

In some embodiments, the first individual augment is used in a joint arthroplasty selected from the group consisting of: total knee arthroplasty, total hip arthroplasty, and total shoulder arthroplasty; or wherein the first individual augment is used to restore bone stock in a bone defect selected from the group consisting of: a femoral bone defect, an acetabular bone defect, a humeral bone defect, a glenoid bone defect, and a periprosthetic bone defect.

In some embodiments, the present invention provides a method, wherein the method restores bone support for an implant for joint arthroplasty, wherein the method comprises: a. assessing a defect of a bone, wherein the bone defect has a first and second void; b. selecting a first individual augment from a kit, wherein the thickness of the first individual augment is sufficient to fill the first void; c. preparing a first surface of the bone associated with the first void; d. selecting a second individual augment from the kit, wherein the thickness of the second individual augment is sufficient to fill the second void; e. preparing a second surface of the bone associated with the second void and offset from the first surface by the difference between the thickness of the first individual augment and the thickness of the second individual augment, f implanting the first individual augment within the first surface of the bone, and implanting the second individual augment within the second surface of the bone so the sides configured to contact an implant of the first and second individual augments are substantially co-planar; g. positioning an implant on top of the sides configured to contact an implant of the first and second individual augments; and h. fixing the implant to the sides configured to contact an implant of the first and second individual augments, wherein the exterior profiles of the first and second individual augments together substantially match an exterior profile of the implant.

In some embodiments, bioactive bone graft material is added to the first individual augment, or the second individual augment, or the first and second individual augments.

In some embodiments, the first and second individual augments are used in a joint arthroplasty selected from the group consisting of: total knee arthroplasty, total hip arthroplasty, and total shoulder arthroplasty; or wherein the first and second individual augments are used to restore bone stock in a bone defect selected from the group consisting of: a femoral bone defect, an acetabular bone defect, a humeral bone defect, a glenoid bone defect, and a periprosthetic bone defect.

In some embodiments, the present invention provides a method, wherein the method restores bone support for an implant for joint arthroplasty, wherein the method comprises: a. assessing a defect of a bone, wherein the bone defect has a first void; b. selecting at least one first individual augment from a kit, wherein the thickness of the at least one first individual augment is sufficient to fill the first void and provide a surface for an implant; c. preparing a first surface of the bone associated with the first void; d. implanting the at least one first individual augment within the first surface of the bone; e. positioning the implant on top of the side configured to contact an implant of the at least one first individual augment; and f. fixing the implant to the side configured to contact an implant of the at least one first individual augment, wherein the exterior profile of the at least one first individual augment substantially match an exterior profile of the implant.

In some embodiments, bioactive bone graft material is added to the at least one first individual augment.

In some embodiments, the at least one first individual augment is used in a joint arthroplasty selected from the group consisting of: total knee arthroplasty, total hip arthroplasty, and total shoulder arthroplasty; or wherein the at least one first individual augment is used to restore bone stock in a bone defect selected from the group consisting of: a femoral bone defect, an acetabular bone defect, a humeral bone defect, a glenoid bone defect, and a periprosthetic bone defect.

In some embodiments, the first individual augment is fixed to the bone by one of pegs, pins, posts, buttons, keels, spikes or hooks.

In some embodiments, the at least one first individual augment is fixed to the bone by one of pegs, pins, posts, buttons, keels, spikes or hooks.

In some embodiments, the second individual augment is fixed to the bone by one of pegs, pins, posts, buttons, keels, spikes or hooks.

In FIGS. 2, 4, 5 and 6, the augments are shown as a lateral augment, however it is understood that an augment of the present invention can be sufficiently designed as a medial augment or a full medial/lateral augment (e.g., similar in size and shape to a tibial tray component), which effectively has a posterior surface sized to match a tibial tray.

An augment of the present invention can be placed into contact or connection with both an implant and a bone surface prepared to receive an implant. “Contact” or “connection” according to the present invention is either direct physical interface with an implant and/or a bone surface, or, optionally, indirect interface with an implant and/or bone surface, in which an intermediary substance, such as cancellous bone paste or bone cement, is interposed between the augment and the implant and/or bone surface.

In the event of future revision, by way of example, the surgeon needs to remove the tibial tray implant (50) from the tibia while leaving the osteointegrated augment (100a, 100b, 100c or 100d) in place. It is understood this surgical situation represents an advantage over the conventional technique, as bone loss during revision is going to be greatly reduced by leaving the augment in place, versus what would be seen in other techniques, which requires augment removal.

Implanting an augment of the present invention can be completed using the same basic instrumentation (e.g., resection guide) as the conventional technique. Transitioning from using a conventional metallic augment that is biocompatible but has no biological activity to using an augment of the present invention will provide biological restoration of missing bone, and can advantageously provide an improved bone environment should the joint require additional revision.

According to a first variation, the augment (100a, 100b, 100c or 100d) features overall dimensions dissimilar to the defect. In this event, the surgeon follows the same steps as described above with the exception the surgeon will shape the contour of the augment (100a, 100b, 100c or 100d) to match the defect.

According to a second variation, the augment (100a, 100b, 100c or 100d) can be semi-contained into a housing made from a different material. The surface of the augment (100a, 100b, 100c or 100d) in contact with the receiving bone will not be fully covered by the housing, enabling osteointegration to take place.

An augment of the present invention can be manufactured by an additive technology such as three dimensional printing (3DP) or stereolithography using, for example, Calcium Hydroxyapatite-Tricalcium Phosphate (HA-TCP) powder.

In an embodiment, the porous structure may be produced by Laser Engineered Net Shaping (LENS®), a process where tiny particles are blown into the path of a laser and melted. The material cools and hardens as soon as it is out of the laser beam, and custom parts can be quickly built up layer by layer. The process is so precise that augments can be used straight off the printer without the polishing or finishing needed in traditional manufacturing. The LENS® process allows augments to be manufactured with microscopic holes for bone to grow into and attach. The augments created from LENS® can integrate into the body more effectively, encouraging bone regrowth that ultimately result in a stronger, longer lasting augment. A CT scan or MRI can be used to make a 3-D model of the bone defect.

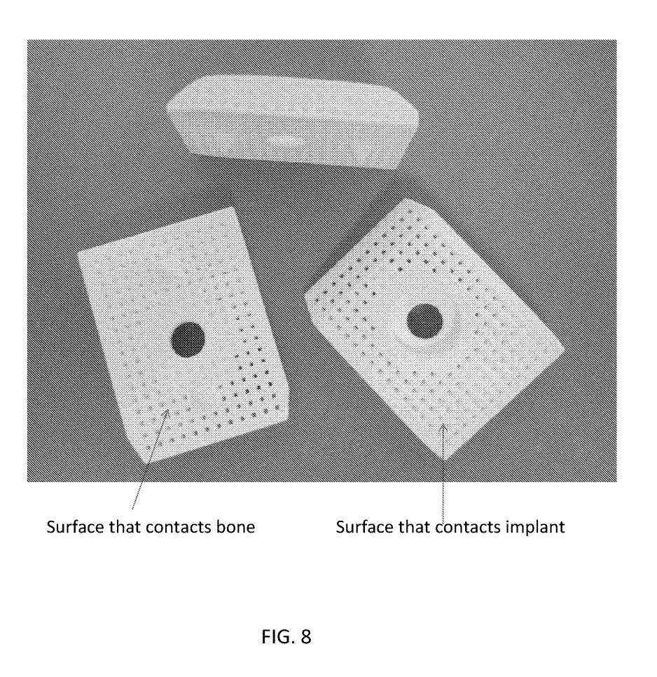

Parts have been successfully manufactured for distal femoral augmentation using an additive technology (see FIGS. 8-10). In FIG. 8, the augment includes a three dimensional endoskeleton support having interconnected macropores providing a framework for host bone to grow into the augment. The augment has a proximal side (sufficiently designed to contact a receiving bone), a distal side (sufficiently designed to contact an implant) and an exterior profile, wherein the proximal side and the distal side are separated by a thickness, wherein the proximal side includes at least one fixation means sufficiently designed to facilitate anchoring of the augment to the receiving bone.

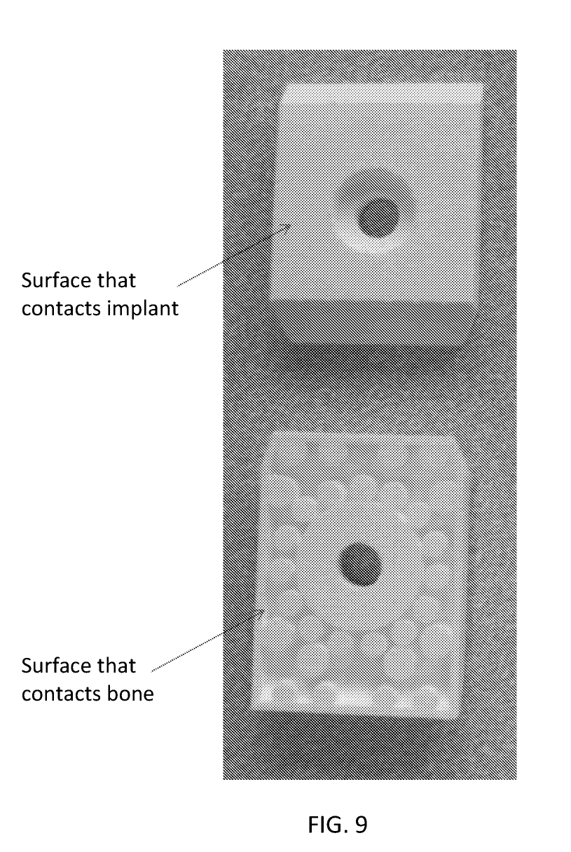

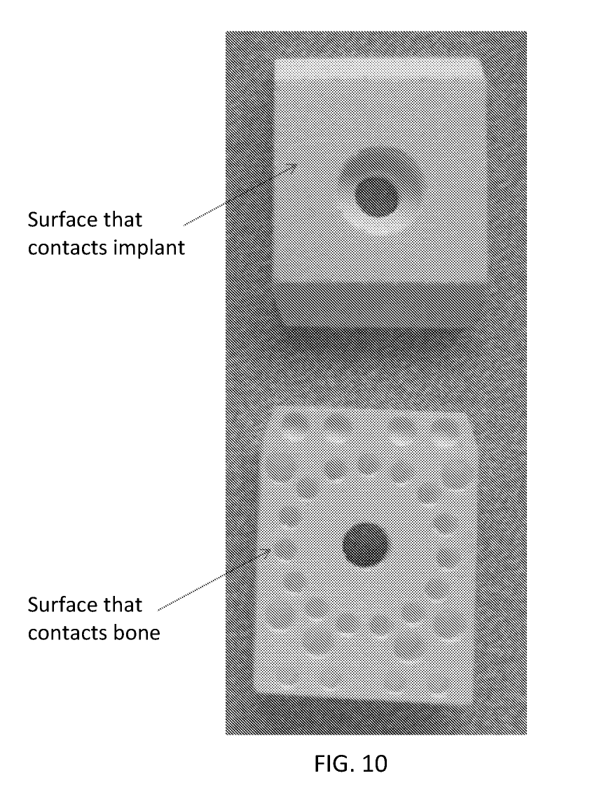

In FIGS. 9 and 10, the augments include a proximal side (sufficiently designed to contact a receiving bone) with a 3D pattern, a solid distal side (sufficiently designed to contact an implant), and an exterior profile, wherein the proximal side and the distal side are separated by a thickness, wherein the augment includes at least one hole for fixing the augment to the receiving bone. The only difference between FIGS. 9 and 10 relates to the 3D pattern of the proximal side. The outside shell aspect is the same for both types. The proximal side is sufficiently designed to be filled with bioactive bone graft.

In another embodiment of the present invention, pre-made augments are provided with a bioactive bone graft component overlying an endoskeleton structure. In this concept, a biologic endoskeleton (as described above) of fixed shape is manufactured, readily available, and provides both structure and bioactive bone graft as a single unit, requiring only attachment to bone. In this concept, the mild modifications to the implanted devices discussed above might be used in conjunction with the implanted metallic and endoskeleton devices to protect the graft and load it properly, while incorporation and remodeling occur. This might be in the form of customizable augments (based upon CT or MRI 3-D reconstructions) or from prefabricated shapes—such as cones and sleeves. This would be designed to fit preformed defects, attach to host bone, and be contoured to fit more standard implanted devices for the hip knee, shoulder, (Cones and or sleeves for the tibia, femur in TKA; augments for the acetabulum, greater trochanter, or femur for THA, glenoid for total shoulder replacement) etc.

In an embodiment, an augment of the present invention can be used during a total knee arthroplasty (TKA). In an embodiment, an augment of the present invention can be used to restore patient bone stock in cases of a tibia bone defect. In an embodiment, an augment of the present invention can be used to restore patient bone stock in cases of a femoral bone defect. In an embodiment, an augment of the present invention can be used during total hip arthroplasty (THA). In an embodiment, an augment of the present invention can be used to restore patient bone stock in cases of an acetabular bone defect. In an embodiment, an augment of the present invention can be used during total shoulder arthroplasty (TSA). In an embodiment, an augment of the present invention can be used to restore patient bone stock in cases of a humeral bone defect. In an embodiment, an augment of the present invention can be used to restore patient bone stock in cases of a glenoid bone defect. In an embodiment, an augment of the present invention can be used to restore patient bone stock in cases of a periprosthetic bone defect. This benefit is achieved by primarily attaching/securing a augment to the bone prior to placement of the implant (rather than primarily attaching/securing a metal augment to the implant, as with the current method).

In an embodiment, an augment of the present invention includes a three dimensional endoskeleton support having interconnected macropores, the macropores providing a framework for host bone to grow into the augment, wherein the support is manufactured from a microporous biomaterial, wherein the support has a distal surface, a proximal surface and an exterior profile defined by a posterior curvature, a lateral curvature, an anterior curvature, and a truncated aspect, wherein the distal surface and the proximal surface are separated by a thickness, wherein the distal surface includes at least one fixation means sufficiently designed to facilitate anchoring of the augment to a receiving bone, and wherein the proximal surface is sufficiently designed to engage a component of an orthopaedic implant.

In an embodiment, a prosthesis system includes a joint prosthesis component; and an augment comprising a distal surface, a proximal surface, and thickness therebetween, at least one of which mimics an exterior profile of the joint prosthesis component, wherein the distal surface includes at least one fixation means sufficiently designed to facilitate anchoring of the augment to a receiving bone, and wherein the proximal surface is sufficiently designed to engage a surface of the joint prosthesis component. In an embodiment, the system further includes bioactive bone graft material.

In an embodiment, a knee prosthesis system includes a tibial tray component having an inferior surface, a superior surface, and an exterior profile; and an augment comprising a distal surface, a proximal surface and an exterior profile defined by a posterior curvature, a lateral curvature, an anterior curvature, and a truncated aspect, at least one of which mimics the exterior profile of the tibial tray component, wherein the distal surface and the proximal surface are separated by a thickness, wherein the distal surface includes at least one fixation means sufficiently designed to facilitate anchoring of the augment to a receiving bone, and wherein the proximal surface is sufficiently designed to engage the inferior surface of the tibial tray component. In an embodiment, the system further includes bioactive bone graft material.

In an embodiment, a method of the present invention includes assessing a bone defect of a tibial bone; implanting an augment within the bone defect so as to create a substantially flush proximal surface, wherein the augment comprises a distal surface, a proximal surface and an exterior profile defined by a posterior curvature, a lateral curvature, an anterior curvature, and a truncated aspect, wherein the distal surface and the proximal surface are separated by a thickness, wherein the distal surface includes at least one fixation means sufficiently designed to facilitate anchoring of the augment into a void created by the bone defect; adding bioactive bone graft material to the augment; positioning a tibial tray component on top of the proximal surface of the augment; and fixing the tibial tray component to the augment.

Publications cited throughout this document are hereby incorporated by reference in their entirety. Although the various aspects of the invention have been illustrated above by reference to the examples and preferred embodiments, it will be appreciated that the scope of the invention is defined not by the foregoing description, but by the following claims properly construed under principles of patent law.

1. An augment, comprising:
a. a side configured to contact a receiving bone having a void;
b. a side configured to contact an implant; and
c. an exterior profile,
wherein the side configured to contact the receiving bone and the side configured to contact the a implant are separated by a thickness; and
wherein the augment is configured to anchor to the receiving bone at a surface of the receiving bone associated with the void so as to restore or partially restore a native shape of the receiving bone.

2. The augment of claim 1, wherein the augment is manufactured from a material selected from the group consisting of: a metal biomaterial, a ceramic biomaterial, a composite biomaterial, and a polymer biomaterial.

3. The augment of claim 1, wherein the augment is resorbable.

4. The augment of claim 1, wherein the side configured to contact the receiving bone further comprises a three dimensional endoskeleton support having interconnected macropores.

5. The augment of claim 4, wherein the support is manufactured from a material selected from the group consisting of: a synthetic biomaterial, a combination of a natural and synthetic biomaterial, and a resorbable biomaterial.

6. The augment of claim 1, wherein at least a portion of the exterior profile is has the same configuration of a least a portion of an exterior profile of the implant.

7. A kit, comprising a joint prosthesis component and a plurality of the augments of claim 1, wherein an individual augment of claim 1 within the plurality differs in at least one property from the other individual augments of claim 1 within the plurality, wherein the at least one property is selected from the group consisting of: thickness and exterior profile.

8. The kit of claim 7, further comprising bioactive bone graft material.

9. A method, wherein the method restores bone support for an implant for joint arthroplasty, wherein the method comprises:
a. assessing a defect of a bone, wherein the bone defect has a first void;
b. preparing a first surface of the bone not associated with the first void;
c. selecting a first individual augment from a kit of claim 7, wherein the thickness of the first individual augment is sufficient to fill the first void;
d. preparing a second surface of the bone associated with the first void and offset from the first surface by the thickness of the first individual augment;
e. implanting the first individual augment within the second surface of the bone so the side configured to contact an implant of the first individual augment is substantially co-planar with the first surface of the bone to provide a reconstructed flat surface;
f. positioning an implant on top of the reconstructed flat surface; and
g. fixing the implant to the bone and the side configured to contact an implant of the first individual augment.

10. The method of claim 9, wherein a bioactive bone graft material is added to the first individual augment.

11. The method of claim 9, wherein the first individual augment is used in a joint arthroplasty selected from the group consisting of: total knee arthroplasty, total hip arthroplasty, and total shoulder arthroplasty; or wherein the first individual augment is used to restore bone stock in a bone defect selected from the group consisting of: a femoral bone defect, an acetabular bone defect, a humeral bone defect, a glenoid bone defect, and a periprosthetic bone defect.

12. A method, wherein the method restores bone support for an implant for joint arthroplasty, wherein the method comprises:
a. assessing a defect of a bone, wherein the bone defect has a first and second void;
b. selecting a first individual augment from a the kit of claim 7, wherein the thickness of the first individual augment is sufficient to fill the first void;
c. preparing a first surface of the bone associated with the first void;
d. selecting a second individual augment from the kit of claim 7, wherein the thickness of the second individual augment is sufficient to fill the second void;
e. preparing a second surface of the bone associated with the second void and offset from the first surface by the difference between the thickness of the first individual augment and the thickness of the second individual augment,
f. implanting the first individual augment within the first surface of the bone, and implanting the second individual augment within the second surface of the bone so the sides configured to contact an implant of the first and second individual augments are substantially co-planar;
g. positioning an implant on top of the sides configured to contact an implant of the first and second individual augments; and
h. fixing the implant to the sides configured to contact an implant of the first and second individual augments,
wherein the exterior profiles of the first and second individual augments together substantially match an exterior profile of the implant.

13. The method of claim 12, wherein a bioactive bone graft material is added to the first individual augment, or the second individual augment, or the first and second individual augments.

14. The method of claim 12, wherein the first and second individual augments are used in a joint arthroplasty selected from the group consisting of: total knee arthroplasty, total hip arthroplasty, and total shoulder arthroplasty; or wherein the first and second individual augments are used to restore bone stock in a bone defect selected from the group consisting of: a femoral bone defect, an acetabular bone defect, a humeral bone defect, a glenoid bone defect, and a periprosthetic bone defect.

15. A method, wherein the method restores bone support for an implant for joint arthroplasty, wherein the method comprises:
a. assessing a defect of a bone, wherein the bone defect has a first void;
b. selecting at least one first individual augment from a the kit of claim 7, wherein the thickness of the at least one first individual augment is sufficient to fill the first void and provide a surface for an implant;
c. preparing a first surface of the bone associated with the first void;
d. implanting the at least one first individual augment within the first surface of the bone;
e. positioning the implant on top of the side configured to contact an implant of the at least one first individual augment; and
f. fixing the implant to the side configured to contact an implant of the at least one first individual augment,
wherein the exterior profile of the at least one first individual augment substantially match an exterior profile of the implant.

16. The method of claim 15, wherein a bioactive bone graft material is added to the first individual augment.

17. The method of claim 15, wherein the at least one first individual augment is used in a joint arthroplasty selected from the group consisting of: total knee arthroplasty, total hip arthroplasty, and total shoulder arthroplasty; or wherein the at least one first individual augment is used to restore bone stock in a bone defect selected from the group consisting of: a femoral bone defect, an acetabular bone defect, a humeral bone defect, a glenoid bone defect, and a periprosthetic bone defect.

18. The augment of claim 1, wherein at least one of the side configured to contact the implant or the exterior profile is reversible secured to the implant.

19. The augment of claim 1 further comprising at least one of a leg, pin, post, button, keel, spike, hook or combination of the foregoing to facilitate implanting the augment to the surface of the receiving bone.
